(19) United States
(12) Patent Application Publication
(10) Pub. No.: US 2006/0189850 A1
(43) Pub. Date: Aug. 24, 2006

(54) Flexible membrane device for measuring at least one physiological information with, corresponding sensor module and method

(76) Inventors: Philippe Cosquer, Trebeurden (FR); Clement Guezou, Lannion (FR); Bruno Dooval, Lannion (FR); Stephane Bruno, Lannion (FR)

Correspondence Address: Gerald E Helget, Suite 2200, 80 South Eighth Street, Minneapolis, MN, 55402, US

(21) Appl. No.: 10/550,665

(22) PCT Filed: Mar. 25, 2004
(86) PCT No.: PCT/FR04/00761

§ 371 Date: Mar. 17, 2006

(30) Foreign Application Priority Data
Mar. 25, 2003 (FR) 0303804

(51) Int. Cl. A61B 5/00; A61B 5/02; A61B 5/08; A61B 5/103

(52) U.S. Cl. 600/300; 600/549; 600/485; 600/500; 600/529; 600/587

(57) ABSTRACT

The invention concerns a device for measuring at least one item of physiological information in an individual, comprising a flexible membrane designed to come into contact with the skin of the individual and participating in the definition of a deformable space for a flexible substance, the substance transmitting to at least one sensor at least one physical force undergone by the membrane.

### BACKGROUND OF THE INVENTION

The field of the invention is that of the determination and use of one or more items of physiological information representing the state of a human or animal subject.

More precisely, the invention concerns self-contained devices, at least with regard to the recording of force measurements, capable of supplying physiological information and corresponding decisions, for example in order to signal an abnormal situation or to indicate a sleep phase.

A particular field of the invention is that of the detection of certain abnormal situations, especially pathological, and in particular falls, in a human subject. The aim of such of detection is in particular the transmission of an alarm to a third person (a physical person or services, etc) fulfilling a remote monitoring function.

The invention can find applications in many situations, and can in particular equip aged persons and/or those with reduced mobility, isolated workers, children, animals, etc.

Known remote alarm systems generally consist of a unit carried by the subject and connected to a fixed base, for example by an HF link. In the event of an abnormal situation, the fixed base sends a coded message, conveyed for example by the telephone network, to a specialist centre. In certain institutions, the message may be conveyed by an internal network.

In all cases, these systems provide for an intentional triggering of the process after the event, following a fall or malaise. This therefore assumes that the subject has the capability and the will to do it.

Various remote-alarm systems are already known. In particular, there exist devices carried in the form of a medallion around the neck, which require a gripping or pulling action to emit an alarm. Apart from the fact that this is a case of passive systems (requiring intentional action by the wearer) these devices often produce false alarms, for example during the night, when they are worn and are unintentionally caught up in the bedding. In addition, it is not known when they are not being worn.

Movement sensors are also known, which give information about abnormal nocturnal activity, or position sensor systems equipping the beds in certain medical services.

In this case, the device of the invention is worn by a user, and can, possibly at a distance, inform a third party in the case of an abnormal situation.

Such a device is in particular described in the patent document FR-2 808 609. This device can be in the form of a watch bracelet.

As illustrated in FIG. 1, the watch casing device 11 is held on the wrist by means of an elastic bracelet 12, similar to that of a conventional watch. The heartbeat 13 passes through the vessels 14, the muscle, the tissues 15 and the skin and then propagates through the bracelet 12, which generates a force on a sensor 16.

Thus the physiological parameters accessible at the wrist are the image of the heartbeat, from which it is possible to deduce the pulse and respiratory frequency, and where applicable the blood pressure.

By placing a temperature sensor very close to the skin, the local temperature is also obtained.

Another particular field of the invention is that of sleep analysis, and in particular the recognition and quantification of the various sleep phases in the subject. In this case, the invention concerns a self-contained portable device for recognizing sleep phases, which uses the same means, or similar means, the analysis of the measurements being adapted accordingly.

The applications of the recognition and quantification of sleep phases are many. They relate to the medical field (aid to the diagnosis of sleep apnoea in pneumology, sleep disturbances, etc), and the “general public” (evaluation of the quality and quantity of sleep in a person, or waking in preselected phase).

One drawback of this device is that it is relatively complex mechanically, because of the presence of the piston, and that it may not be easy to assemble. It assumes a relatively large number of parts, grouped together in a small space.

In addition, the presence of a contact placed against the skin of the wearer may prove to be uncomfortable, because of its rigidity and its material or texture.

Another drawback is that the piston may, in certain situations, function insufficiently, in particular because it moves in only one direction.

Finally, because of the structure of the shell+piston system, the shell may in certain cases come into abutment against a bone, then altering or even preventing the measurement.

### SUMMARY OF THE INVENTION

A device for measuring at least one item of physiological information in an individual, comprising a flexible membrane, designed to come into contact with the skin of the said individual and participating in the definition of a deformable space for a flexible substance, the said substance transmitting, to at least one sensor, at least one physical force undergone by the said membrane. The said deformable space is defined by a support (for example a printed circuit) on which the said sensor or sensors and the said membrane are mounted, so that the said substance is in direct contact with the said sensor or sensors.

An object of the invention is in particular to mitigate these various drawbacks of the prior art.

More precisely, an objective of the invention is to provide a device for measuring at least one item of physiological information, which is simple to manufacture and requires a small number of parts.

Another objective of the invention is to provide such a device which is more comfortable for the user to wear.

Yet another objective of the invention is to provide such a device which is more effective than the known systems.

Another object is it is thus possible to omit the piston, and the rigid element in contact with the skin of the wearer. The membrane and the substance it contains are deformable, or flexible, the space (or cavity) occupied by the substance therefore itself being able to deform so as to allow a new distribution of the volume under the effects of a force, which can be detected by sensors.

Another object is that a simple and effective system is therefore obtained. In particular, the sensing surface covers a large area, avoiding any risk with respect to contact with a bone (the membrane following the surface of the skin).

Another object is that the membrane comprises means of fixing to the said support. In particular, the membrane can define at least one housing designed to receive the said support, allowing rapid and effective fixing. It may for example be a case of at least one groove, a notch and/or a recess.

Another object is that the membrane advantageously comprises means of fixing to a shell element of the said device. The membrane preferentially defines at least one housing designed to receive the shell element. It may for example be a case of at least one groove, a notch and/or a recess. These fixing means act by clipping, so as to allow easy and effective mounting.

According to another advantage aspect of the invention, the said membrane has at least two areas with different rigidities. The membrane can advantageously have a main contact area, designed to come into contact with the skin of the said individual, and a peripheral area, extending over the contour of the said main contact area. Each of the said areas fulfils a distinct function, belonging to the group comprising the measurement of forces, the transmission of forces and the rigidity of the shape of the said membrane. According to a first advantageous embodiment of the invention, the thickness of the said peripheral area is less than the thickness of the said main contact area.

Another advantage is the membrane is obtained by overmoulding at least two materials with different rigidities. Preferentially, the said membrane is produced in at least one hypoallergenic material.

Another advantageous characteristic of the invention, the membrane and/or the said substance has an elastic character. This makes it possible to return the assembly to a position of equilibrium. For example, the membrane can have a flexibility of around 60 Shore A.

It is also advantageous, in order for the volume of the cavity to remain substantially constant, for the said substance to be a non-compressible or only slightly compressible material. This substance has an elasticity advantageously chosen so as to optimise a compromise between the speed of return to the initial position of the said membrane and the amplitude of the resulting oscillation.

In one advantageous embodiment, the substance is a dielectric material. In another particular embodiment, the said substance is a silicone gel.

According to yet another advantageous aspect of the invention, the device comprises at least one transducer for measuring at least one dynamic force, representing a blood pressure wave and/or a relative movement. Alternately or in a complementary fashion, the device comprises at least one transducer for measuring at least static force is provided.

Advantageously, it also comprises a sensor for the temperature of the substance representing the skin temperature of the user.

Another object according to a preferential embodiment, the sensor or sensors comprise at least one piezocapacitive sensor and/or at least one piezoresistive sensor and/or at least one contact switching at a predetermined pressure.

Another object is the support is a printed circuit carrying electronic components in order to reflect the amplification, treatment and processing of electrical signals and/or a decision relating to a state of the wearer, means for supplying electrical energy and/or a communication interface.

Advantageously, the device comprises a shell formed by two complementary shell elements, a bottom shell element carrying the said membrane and a top shell element.

Another object and advantage is the shell elements are fixed together by screwing and/or clipping and/or adhesive bonding, also providing a seal for the said fluid.

Another object and advantage is two types of seal are provided: the fluid must be kept in its cavity and the device must be watertight (bath and shower). Preferentially, the properties of the membrane and of the assembly are used to achieve this. Screws are there to compress the flexible areas and guarantee the impermeability of the whole. Mounting by clips or adhesive bonding can also be envisaged.

Another object is that the device has a holding strap, fixed to the top shell element. The holding strap and/or the top shell element has capacity for extension and elastic recovery, so as to facilitate the application of prestressing to the device. The holding strap and at least one portion of the shell can advantageously form a single piece produced from a flexible material.

Another object and advantage is that the invention can in particular comprise processing means analyzing at least one physical force transmitted by the said fluid in order to determine at least one of the items of information belonging to the group comprising:

at least one item of blood pressure information;

at least one item of information representing the pulse;

at least one item of information representing arterial tension;

at least one item of information representing respiration;

at least one item of information representing the activity of the said individual;

at least one item of information representing a fall;

at least one item of information representing the wave form;

at least one item of information representing the skin temperature of the wearing area;

at least one item of information as to whether the device is worn/not worn;

at least one item of information representing the change and/or variance of one of the said above items of information.

This makes it possible to achieve a very large number of applications.

Another object and advantage is that the invention also concerns a sensor module per se, intended to equip or cooperate with a device as described above. Such a module comprises a flexible membrane, designed to come into contact with the skin of the said individual and participating in the definition of a deformable space for a flexible substance, the said substance transmitting to at least one sensor at least one physical force undergone by the said membrane. It may for example then be attached in a casing of the wristwatch type.

Another object and advantage of the invention also concerns the method of manufacturing such a device for measuring at least one item of physiological information. This method advantageously comprises the following steps:

mounting the necessary electronic components on a support;

connecting together the membrane and the support, defining a deformable space; and

injecting the said substance in the said space.

Another object according to one advantageous embodiment, the substance is injected into the said space in a liquid form.

Another object and advantage preferentially, the support is inserted in at least one housing defined in the membrane, the membrane is connected to a bottom shell element, by means of at least one housing provided for this purpose on the membrane (self-holding).

The method advantageously comprises a step of assembling a shell from a bottom shell element and a top shell element. The shell elements can in particular be connected together by screwing and/or clipping and/or adhesive bonding.

Other characteristics and advantages of the invention will emerge more clearly from a reading of the following description of a preferential embodiment of the invention, given by way of simple illustrative and non-limiting example, and the accompanying drawings.

### BRIEF DESCRIPTIONS OF THE DRAWINGS

FIG. 1 presents a device according to the prior art, already commented on in the background;

FIGS. 2A and 2B are two overall views of a device according to the invention (without the bracelet);

FIGS. 3A and 3B are two exploded views of the device embodiments of FIGS. 2A and 2B;

FIG. 4 is a view of an embodiment of a membrane of the device of FIGS. 2A and 2B;

FIG. 5 is a view in section of the membrane in FIG. 4, fixed to a printed circuit;

FIGS. 6 and 7 are enlargements of two elements of the section in FIG. 5;

FIG. 8 illustrates in a simplified fashion a method of manufacturing a device of FIGS. 2A and 2B.

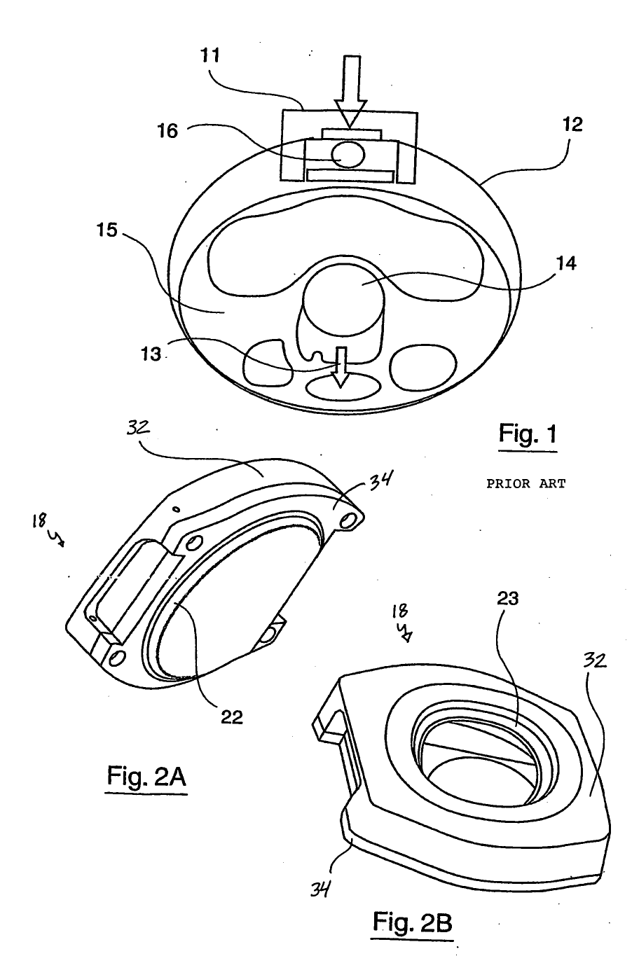

### DETAILED SPECIFICATION

The invention is therefore based on the use of flexible materials, making it possible to analyze one or more items of physiological information. These can then be analyzed, in the context of several applications, such as for example the detection of a fall or abnormal situations, or monitoring sleep phases.

The embodiment described hereinafter concerns an intelligent multi-sensor assembly (“smart device”) making it possible to measure, process and analyze physiological phenomena such as the blood pressure wave, which is a source of several vital items of information, and in particular the pulse, the respiratory frequency and the blood pressure, as well as the associated variances and the waveform. This device also makes it possible to detect movements representing the activity of the user, or wearer, and the skin temperature, etc.

As can be seen in FIGS. 2A and 2B, the device 18 of the invention can be produced in the form of a casing 32 similar to that of a conventional watch, at least with regard to its shape and dimensions. As will be seen subsequently, this device 18 comprises a casing 32, a membrane 22 and a transparent top surface 23, allowing the display of information, for example in the form of a liquid crystal screen. This information can in particular include conventional information found on a watch, as well as physiological information, or corresponding alarms.

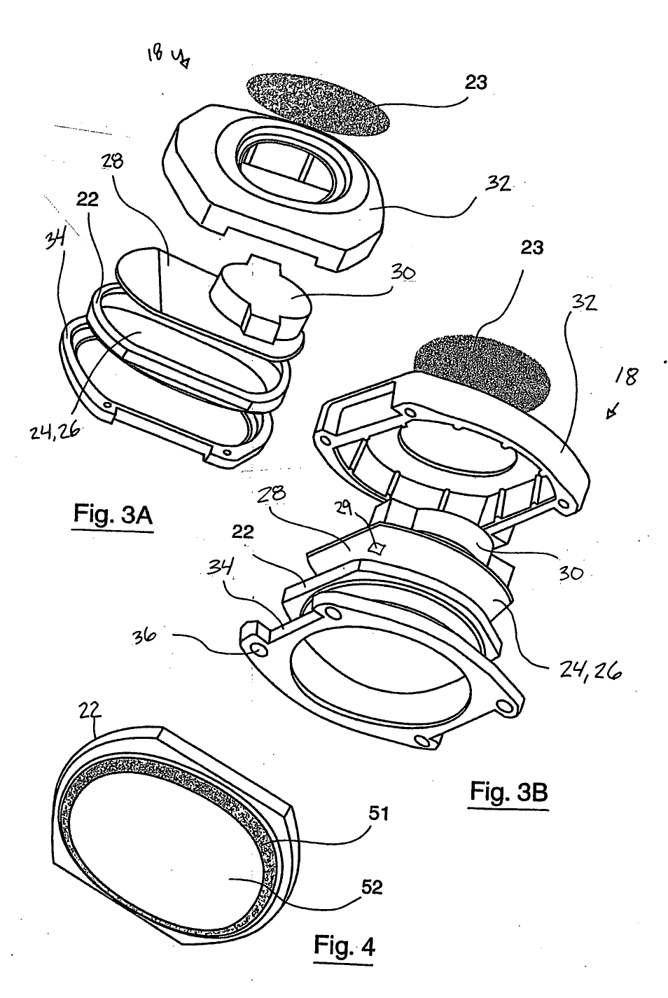

The use of the device 18 of the invention is therefore based on four main units, which appear more clearly in the exploded views in FIGS. 3A and 3B;

a flexible membrane 22;

a substance 24, or a fluid, for the transmission of physical quantities to transducers 29, this fluid 24 being confined in a space 26 defined by the membrane 22;

transducers 29, mounted on an electronic or support card 28 (or support or printed circuit) carrying the various electronic amplification and filtering components necessary for the processing of signals, the processing unit, the communication interfaces and the means delivering electrical energy (battery housed in the housing 30);

a mechanical subassembly 32 and 34 and a holding strap (not shown).

The first three units 22, 24, 28, 30 form the intelligent sensor assembly 31, which is encapsulated in the last unit 32.

FIGS. 3A and 3B show the arrangements of these various units, according to one advantageous embodiment of the invention.

The flexible membrane 22 forms, with the electronic support 24, after assembly, a deformable cavity or space 26. The bottom shell element 34 is fixed to the membrane 22, as will be seen subsequently, so as in particular to hold the assembly 31 temporarily.

The substance, or fluid 24, is then injected at the initial liquid stage into this cavity, through an orifice provided for this purpose. This fluid 24 will make it possible to transmit the external physical forces to the transducers 29, mounted on the electronic card 24, on the space or cavity side, or taking the information from this cavity 26. This fluid becomes, at the final stage, in accordance with the characteristics stated subsequently, and also to a certain extent reinforces the holding of the assembly.

This subassembly, formed from the membrane 22, the electronic unit 28, the fluid 24 at the final stage, and possibly the bottom shell 34, forms the essential part of the invention, or sensor module 18, which can then be put in place in top shell or casing 32. This assembly, once inserted in the top shell 32, is centered by means of the membrane 22, which also procures impermeability of the assembly, once assembled.

The anchoring of the holding strap (not shown) is preferentially effected on the top shell 32. These two elements form the fixing unit. Screws, through threaded apertures 36, hold the sensor module to this fixing unit. They also guarantee slight compression of the top part of the membrane, thus ensuring impermeability of the assembly.

Other types of connection, for example by clipping or adhesive bonding, can of course be envisaged in addition or alternatively.

According to the invention, the volume of the cavity 26 defined by the membrane 22 and the support 28 remains constant or practically constant and may deform under the effect of an external mechanical force. The assembly 31 formed by the membrane 22 and the substance 24 must therefore have a deformable property. In this case, there exists a transfer of energy between the external forces and the assembly 31 formed by the membrane 22 and the substance 24, giving rise to the appearance of phenomena perceptible to the transducers.

The substance 24 is therefore preferentially non-compressible, or only slight compressible, in order not to absorb some of this energy.

In order to allow a new distribution of the volume under the effect of a force, the substance 24 and membrane 22 must be deformable. In addition, it is necessary for at least one of these two elements to be elastic, in order to return to the equilibrium position. This may be either one or the other or both. In the case described below, both are.

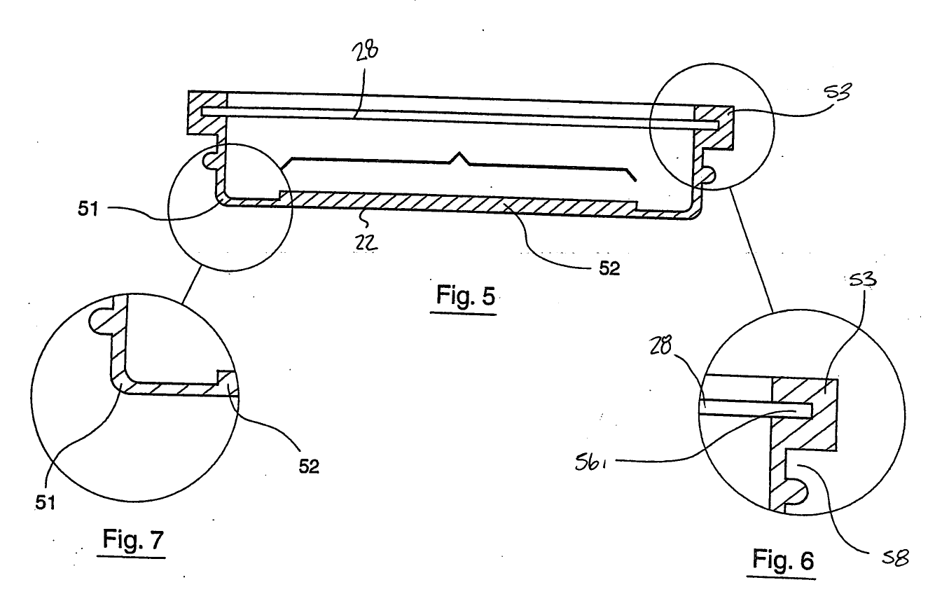

The design of the membrane 22 is illustrated by FIG. 4, and by the corresponding view in section in FIG. 5. FIGS. 6 and 7 are enlargements of two parts of the section in FIG. 5.

This membrane 22, produced from flexible material, and the substance 24 therefore have an essential role in the implementation of the measurement principle. They make it possible in fact to sense the various external mechanical forces (including variations thereof) generated in particular by the arterial (blood) pressure wave and by the relative movements of the device 18 with respect to the body.

These forces are propagated through these two flexible elements 22 and 24 towards the transducers 29 carried by the support 28. It is the properties of the materials that permit the transmission of these external mechanical forces to the transducers 29.

It should therefore be noted that, compared with the prior art, there is no pickup or piston. It is the “flexible belly” of the device 18 that makes it possible to collect the information sought.

The membrane 22 is composed of several areas with different rigidities. In the embodiment illustrated by FIGS. 4 to 7, three zones can be distinguished, each fulfilling a specific function:

a first zone 51 (FIG. 7) which is the most flexible zone, whose function is to allow the external mechanical forces to propagate to the transducers 29. This zone is situated on the periphery of the membrane 22. In the embodiment illustrated, it is a variation in thickness that procures variations in flexibility. This first zone 51 is in this case the finest;

a second zone 52 with a greater rigidity than the first zone 51, making it possible to have a maximum surface area and thus to dispense with the problem of positioning of the device. This zone of greater rigidity prevents excessive deformation of the cavity, which might give rise to poor distribution of the substance 24. It may however be noted that the property of elasticity of the fluid 24 limits this problem;

a third zone 53 (FIG. 6), which makes it possible to fix the membrane 22 to the other elements of the sensor 29. It connects together, through its shape, the electronic support 28 and the membrane 22, by means of the groove 56. This groove 56 achieves the impermeability of the space receiving the substance 24, when the electronic support 24 is inserted in the groove 56. Moreover, this third zone 53 produces the impermeability of the whole device 18 (the sensor unit/shell) by virtue of the slight force exerted by the screws, after the bottom shell element 34 has been fixed to the membrane 22 by means of the recess 58.

The membrane 22 can advantageously be produced at 60 Shore A. Such a membrane gives good results, associated with the fluid 24 mentioned below.

It should be noted that the variation in thickness of the membrane is only one particular embodiment, and a similar result can be obtained, for example using several materials with different rigidities, for example by overmoulding.

The membrane 22 being in direct contact with the skin, it is preferably produced from a hypoallergenic material.

The substance 24 used is advantageously a gel. Its role is to permit the transmission of the mechanical forces, and to keep the membrane 22 in a position of correct functioning, and preferentially to propagate the variations in temperature.

This substance 24 must preferentially be a non-compressible, elastic and chemically stable over time, and have dielectric properties.

Advantageously, this fluid 24 has a liquid character on manufacture, in order to be more easily injected. It then acquires its definitive state within the cavity. This change in characteristic takes place without any emission.

The dielectric character makes it possible not to interfere with the electrical functioning of the electronic unit 28, and to protect it from contact with the membrane 24. Thus the electronic components are effectively protected from short circuits.

The elastic character of the fluid 24 must respect a compromise between the speed of return to the initial position, which must be great, and the aptitude of the resulting oscillation, so as to obtain a critical regime.

One advantageous fluid 24 is a silicone gel. It may in particular be a case of the silicone gel distributed by Dow Corning (registered trade mark) with the reference Sylgard 527 (registered trade mark).

In the embodiment described, the following information is in particular sought:

measurement of dynamic forces (arterial pressure wave and movements);

measurement of static mechanical forces (gripping);

measurement of the temperature.

For measuring the static mechanical force, when it is a case of guaranteeing a minimum static force, the use of a miniature contact with switching at a given pressure (“pressure switch”) may prove suitable. When a more precise measurement is necessary, it will be possible to use a sensor of the piezoresistive type, such as for example the MPX2300D sensor from Motorola (registered trade mark).

The measurement of the dynamic force is preferentially carried out by means of transducers 29 of the piezocapacitive type. This type of transducer 29, associated with a suitable electronic circuit, makes it possible to effectively convert the dynamic and mechanical forces picked up by the sensor 29 unit into information that can be exploited by the processing unit.

The temperature measurement can advantageously be carried out by means of a CTP sensor 29 and an associated electronic circuit or by means of a commercially available integrated sensor such as LM62™ or equivalent. This type of sensor 29, hard-wired on the electronic support 28, will make it possible to measure, to within an inertia, the skin temperature.

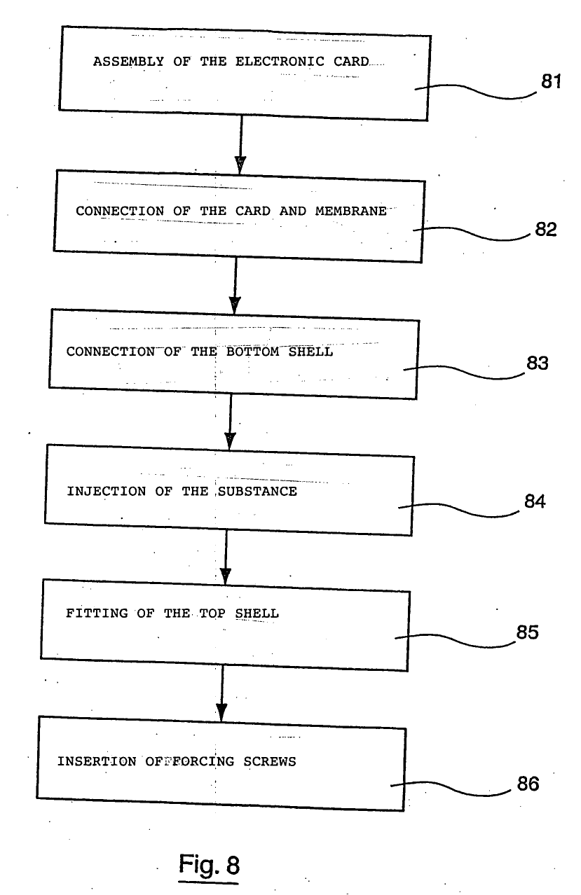

FIG. 8 illustrates, in the form of a simplified flow diagram, an implementation of a method of manufacturing a device 18 according to the invention.

According to this method, the electronic card (81) is first of all assembled, conventionally mounting on it all the necessary electronic components.

Next, this electronic card 28 is fixed (82) to the membrane 22, by means of the groove 56 (FIG. 6). There is then available a sealed space, or cavity 26, defined by this membrane 22 and the electronic card 28. The assembly formed by the membrane and the card are inserted (83) in the bottom shell element 34 which forms a base, and the holding of this assembly is provided by the groove 56.

The substance 24 can then be injected (84), preferably in liquid form, through an opening provided for this purpose, in the housing defined by the membrane 22 and the electronic card 28.

All that then remains to be done is to fit (85) the top shell element 32, or cap. This is easily fixed to the membrane 22, by virtue of the recess 58, provided for this purpose. Finally, forcing screws are attached, which fix to each other the two shell elements 32 and 34, and reinforce the seal by confining the edges of the membrane 22. It should be noted that the operating range of the device is increased when the encapsulation assembly formed by the top shell element 32 and the bracelet has capacity for extensibility and elastic recovery. This makes it possible to easily guarantee the application of a prestressing, which may prove essential for correct functioning. Should it not possess this property, the adjustment and therefore the obtaining of a suitable prestressing may prove particularly tricky to achieve, even if it remains achievable in practice.

According to one advantageous embodiment, two pieces are provided, the top shell element 32 and the bracelet (not shown). According to this embodiment, it is the bracelet that has the property of extensibility. This can of course be limited to a portion of the bracelet. According to another approach, it is possible to provide for the top shell element 32 and the bracelet to form a whole, all or at least part of which, in contact with the skin, would be flexible.

1. A device for measuring at least one item of physiological information through the skin of an individual, comprising:
a) A flexible membrane designed to come into contact with the skin of the individual;
b) A deformable space formed by the flexible membrane;
c) A support card upon which at least one sensor is mounted, the card mounted to the membrane to close the space;
d) A flexible substance filling the space for transmitting to the at least one sensor at least one physical force to be undergone by the membrane.

2. The device of claim 1, further comprising that the membrane comprises means of fixing to the support.

3. The device of claim 2, further comprising that the said membrane defines at least one housing designed to receive the said support.

4. The device of claim 1, further comprising that the said membrane comprises means for fixing to a shell element of the said device.

5. The device of claim 4, further comprising that the said membrane defines at least one housing designed to receive the said shell element.

6. The device one of claim 4, wherein the fixing means acts by clipping.

7. The device of claim 1, wherein the membrane has at least two areas with different rigidities.

8. The device of claim 7, wherein the membrane has a main contact area, designed to come in contact with the skin of the individual, and a peripheral area, extending over the contour of the main contact area.

9. The device of claim 7, wherein each of the areas fulfills a distinct function, belonging to the group comprising the measurement of forces, the transmission of forces and the rigidity of the shape of the membrane.

10. The device of claim 8, wherein the thickness of the peripheral area is less than the thickness of the main contact area.

11. The device of claim 7, wherein the membrane is obtained by overmoulding at least two materials with different rigidities.

12. The device of claim 1, wherein the membrane is produced from at least one hypoallergenic material.

13. The device of claim 1, wherein the membrane and the substance has an elastic character.

14. The device of claim 1, wherein the substance is a substantially non-compressible material.

15. The device of claim 1, wherein the substance is a dielectric material.

16. The device of claim 1, wherein the substance is a silicone gel.

17. The device of claim 1, wherein the sensor is at least one transducer for measuring at least one dynamic force, representing an arterial pressure wave or a relative movement.

18. The device of claim 1, wherein the sensor is at least one transducer for measuring at least one static force.

19. The device of claim 1, wherein the sensor is for the temperature of the said substance, representing the skin temperature of the user.

20. The device of claim 1, wherein the sensor is comprised from a group of a piezocapacitive sensor, a piezoresistive sensor or a contact switching at a predetermined pressure.

21. The device of claim 1, wherein the support card is a printed circuit carrying electronic components for effecting the amplification, treatment and processing of electrical signals and a decision relating to a state of the individual and means for supplying electrical energy and a communication interface.

22. The device of claim 1, further comprising a shell formed from a complementary a bottom shell element carrying the said membrane and a top shell element.

23. The device of claim 22, wherein the shell elements are connected together by screwing, clipping or adhesive bonding, also providing a seal for the substance.

24. The device of claim 22, further comprising a holding strap, fixed to the top shell element.

25. The device of claim 24, wherein the holding strap and the said top shell element has a capacity for extensibility and elastic recovery so as to facilitate the application of a prestressing to the device.

26. The device of claim 25, wherein the holding strap and at least a portion of the shell form a single piece produced from a flexible material.

27. The device of claim 1, further comprising processing means on the support card for analysing at least one physical force transmitted by the substance in order to determine at least one of the items of information belonging to the group comprising:
at least one item of blood pressure information;
at least one item of information representing a pulse;
at least one item of information representing an arterial tension;
at least one item of information representing respiration;
at least one item of information representing an activity of the said individual;
at least one item of information representing a fall;
at least one item of information representing a wave form;
at least one item of information representing the skin temperature of the wearing area;
at least one item of information as to whether the device is worn or not worn; and
at least one item of information representing a change of one of the above items of information.

28. (canceled)

29. A Method of manufacturing a device for measuring at least one item of physiological information comprising the following steps:
mounting the necessary electronic components on a support;
connecting together a membrane and the support, defining a deformable space; and
injecting a substance in the said space.

30. The method of claim 28, wherein the substance is injected into the space in a liquid form.

31. The method one of claim 28, wherein the support is inserted in at least one housing defined in the membrane.

32. The method of claim 28, wherein the membrane is fixed to a bottom shell element, by means of at least one housing provided for this purpose on the membrane.

33. The method of claim 28, further comprising a step of assembling a shell formed from a bottom shell element and a top shell element.

34. The method of claim 32, wherein the shell elements are connected together by screwing, clipping or adhesive bonding.
